(19) United States
(12) Patent Application Publication
(10) Pub. No.: US 2006/0029976 A1
(43) Pub. Date: Feb. 9, 2006

(54) Method for the detection of C-reactive protein in mammalian fluids

(76) Inventors: Jerry K. McVicker, Boone, IA (US); Glenda C. Rouse, Boone, IA (US); Denny M. Barrantes, Scarborough, ME (US)

Correspondence Address: STURM & FIX LLP, 206 SIXTH AVENUE, SUITE 1213, DES MOINES, IA, 50309-4076, US

(21) Appl. No.: 10/913,082

(22) Filed: Aug. 6, 2004

(51) Int. Cl. G01N 33/53

(52) U.S. Cl. 435/7.1

(57) ABSTRACT

A method of determining if the concentration of CRP in biological fluids of a mammal is above a predetermined relative risk level indicative of future cardiac problems. The method includes the steps of taking a sample of biological fluid and applying the sample to a position on a protein binding membrane. The sample is exposed to a dehydrated anti-CRP complexing agent coupled with labeling agent immobilized on the membrane to yield a conjugate which flows along the membrane by wicking action. If the CRP concentration in the sample is high enough, it binds to the conjugate as well as an anti-CRP antibody immobilized at a test position on the membrane resulting in color change at the test position.

### BACKGROUND OF THE INVENTION

1. Field of the Invention

This invention relates generally to C-reactive protein (CRP) testing and more particularly to a method of detecting CRP in mammalian fluids using an immuno assay device.

2. Description of the Related Art

The numbers in brackets refer to the publications listed in the Appendix, the teachings of which are incorporated herein by reference.

Based upon experimental evidence, researchers have recently begun to accept that low-level vascular inflammation is a critical component in atherothrombosis and subsequent coronary heart disease.[1] This represents an enormous change in thinking and has led to many advances in the diagnosis of coronary heart disease. For instance, studies have shown that circulating levels of inflammatory markers, such as C-reactive protein (CRP), rise in individuals at risk for cardiovascular events. [2-4] In fact, several large-scale prospective epidemiological studies have shown that plasma levels of CRP are a strong independent predictor of risk of myocardial infarction, stroke, peripheral arterial disease, and vascular death among individuals without known cardiovascular disease. [4-14]

Although epidemiological studies demonstrate association between low-grade inflammation and vascular risk, the utility of CRP testing appears to be dependent on dividing the CRP values into risk groups.[1] On the basis of available evidence, the American Heart Association and the Centers for Disease Control have defined three such risk groups as set forth in Table 1. [16]

TABLE 1
The AHA/CDC Risk Groups

| Relative Risk | Concentration of CRP (mg/L) |
|---|---|
| Low | <1.0 |
| Average | 1.0 to 3.0 |
| High | >3.0 |

Nevertheless, optimization of tests for this marker will be required to maximize the utility of this marker for risk assessment because traditional CRP tests are designed to monitor acute and chronic inflammation resulting in an inadequate sensitivity for risk stratification. [15]

While the elevation in serum CRP has been shown to be a very good predictor of future cardiovascular events, the rise in CRP levels is relatively low (˜1.5 mg/l), making it difficult to quantitate using conventional clinical chemistry analyzers and test methods. This has led to the development of high-sensitivity CRP (hs-CRP) test methods using enhanced detection methods such as antibody-conjugated latex particles.

The hs-CRP test methods represent a great improvement in sensitivity, however, they are still time-consuming and expensive test methods, especially when used as a screening technique.

### BRIEF SUMMARY OF THE INVENTION

Briefly stated, the present invention provides a method of determining if the concentration of CRP in biological fluids of a mammal is above a predetermined relative risk level indicative of future cardiac problems. The method includes the steps of taking a sample of biological fluid and applying the sample to a position on a protein binding membrane. The sample is exposed to a dehydrated anti-CRP complexing agent coupled with labeling agent immobilized on the membrane to yield a conjugate which flows along the membrane by wicking action. If the CRP concentration in the sample is high enough, it binds to the conjugate as well as an anti-CRP antibody immobilized at a test position on the membrane resulting in color change at the test position.

### BRIEF DESCRIPTION OF THE SEVERAL VIEWS OF THE DRAWINGS

FIG. 1 is a perspective view of a lateral flow device with portions cutaway to show the internal components; and

FIG. 2 is a perspective view of a lateral flow device similar to that shown in FIG. 1, but having multiple test lines.

### DETAILED DESCRIPTION OF THE INVENTION

The present invention provides a lateral flow immunoassay device that can be used as a screening method. The device can be used patient-side, without the need for any additional analytical equipment, and yields test results in less than 20 minutes.

While the lateral flow device looks like devices used in methods previously patented by Applicants (U.S. Pat. Nos. 6,660,534 and 6,245,577), it uses different reagents and is specific for the human C-reactive protein rather than the immunoglobulins of non-human species Other differences include no set cut-off concentration of the target protein, and it is a positive assay rather than an inhibitory assay.

With this device, human CRP is detected by an affinity purified goat anti-CRP antibody bound to gold colloid. The detection limit, or sensitivity of the device, is controlled by dilution of the serum sample. When the diluted sample is applied to the sample well, it re-hydrates the anti-CRP-colloidal gold conjugate and begins flowing through the nitrocellulose strip by wicking action. If the CRP concentration in the sample is high enough, it binds to the conjugate as well as an anti-CRP antibody immobilized on a fixed position on the nitrocellulose strip. The binding results in the visualization of a red line at the test (T) position. The current detection limit is 1.5 mg/L.

The lateral flow device can quickly determine if CRP values are above levels that have been shown to be valuable indicators of future cardiac problems. This could be a very helpful device for screening individuals at risk. In Japan, between the years 1970 and 1996, the increase in acute myocardial infarctions has increased 93.4% per 100,000 people. A recent study in one Danish county of 340,000 inhabitants found that using a screening test, as opposed to full laboratory testing, saved $111,160 per year. The need for an effective screening test exists and the present invention provides a unique test that addresses this need.

Kit Contents

The kit consists of the following;

1. One lateral flow cassette enclosed in a foil wrapper with desiccant

2. One vial of dilution buffer

Materials and Description of the Lateral Flow Device

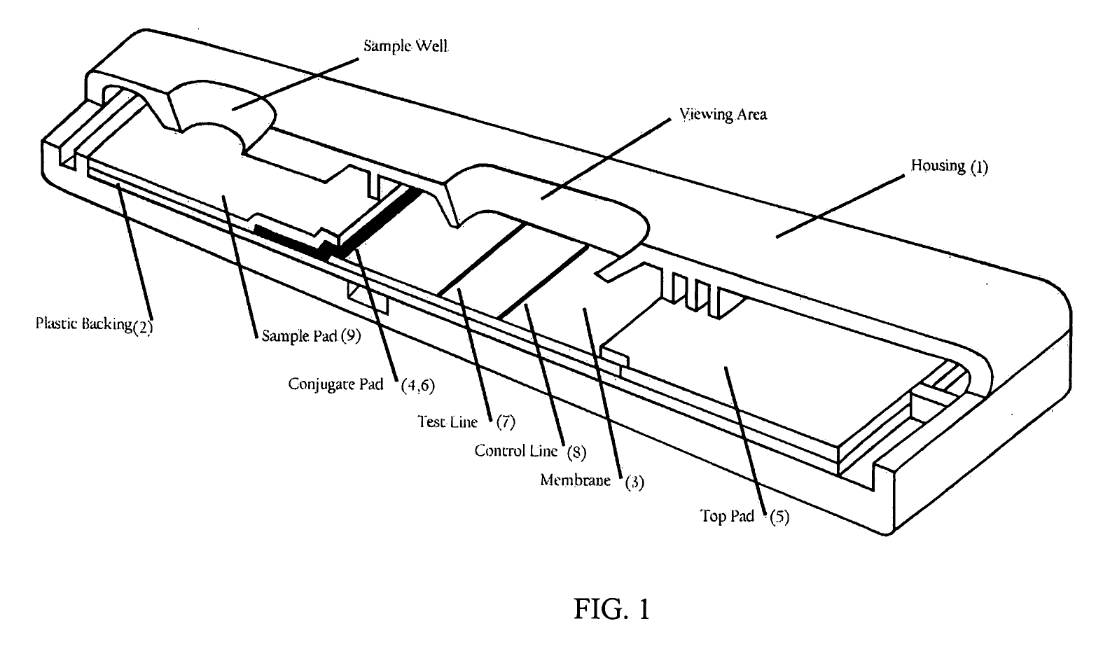

FIG. 1 is a cutaway view of the lateral flow device including the following components:

1. Lateral flow plastic housing: BioSigma or equivalent.

2. Plastic backing (polyester, 100-400 μm thick): Ahlstrom or equivalent.

3. Membrane (Nitrocellulose): Millipore grade HF120 or equivalent.

4. Conjugate release pad: Ahlstrom polyester grade 2033 or equivalent.

5. Top Pad: Schleicher & Schuell grade 470 or equivalent.

6. Colloidal gold labeled goat anti-CRP: Midland BioProducts Corporation or equivalent, particle size range 10-65 nm. Sprayed on conjugate release pad @3 μl/cm (OD_{520 nm}=10) or equivalent.

7. Goat anti-CRP: Midland BioProducts Corporation catalog number 73307 or equivalent. Immobilized on membrane at T (test) position @1.0 mg/ml or equivalent.

8. Protein A: Sigma or equivalent. Immobilized on membrane at C (control) position @1.0 mg/ml or equivalent.

9. Dilution buffer: 0.85% sodium chloride, 0.1% sodium azide or equivalent.

10. Desiccant: Multisorb Technologies or equivalent.

### EXAMPLE 1

The format to detect CRP involves a dry chemistry principle. A small amount of biological fluid (e.g. blood or urine) is collected and mixed with the buffer diluent. A portion of the diluted sample is transferred to the lateral flow cassette and applied at the S (sample) position. The addition of the diluted sample rehydrates the colloidal gold labeled anti-CRP in the conjugate release pad. This allows CRP, if present in the sample, to bind with the colloidal gold labeled anti-CRP.

If the sample contains CRP, a complex will form with the colloidal gold labeled anti-CRP. The complex will flow through the membrane and bind to the immobilized anti-CRP at the T position resulting in the formation of a visible red line. Excess colloidal gold labeled anti-CRP will continue flowing through the membrane and bind to the immobilized protein A at the C (control) position resulting in the formation of a visible red line.

If the sample does not contain any CRP, no complex will form with the colloidal gold labeled anti-CRP. Consequently, no visible red line will form at the T position. However, residual colloidal gold labeled anti-CRP will bind with the immobilized protein A at the C (control) position resulting in the formation of a visible red line.

Sensitivity and thus detection limits can be controlled by adjusting the sample dilution. For example, a dilution of 1/60 results in a detection limit of 1.5 mg/L using the parameters here. While the dilution and detection limit is not a linear relationship, it can be determined experimentally.

Alternate Device Designs

Alternate 1

The test could be applied to other mammals as well (including but not limited to cattle, horses, pigs, dogs, cats). The only changes that would be required would be the species specific of the anti-CRP antibody. For instance, if used with dogs, a polyclonal anti-dog CRP would be used.

Alternate 2

The test could incorporate protein G rather than protein A in the assay control position. Protein G and protein A have similar binding characteristics and are often used interchangeably. The test could also use a mixture of protein A and protein G which is commercially available.

Alternate 3

The test could use native or recombinant forms of the protein G or protein A. These are frequently used interchangeably.

Alternate 4

Cosmetic changes to the test could include any of the following:
Color of the lines
Color of the background
Shape of the lines
Shape of the background
Shape of the cassette
More than one test per cassette
Transposition of the control and test lines
Elimination of the control line

Alternate 5

The nitrocellulose membrane could be changed to any membrane filter with adequate protein binding capacities (e.g. polyvinylidene difluoride, mixed cellulose ester). Currently, the test requires a minimum of 20 μg/cm² protein binding capacity. In addition, the membrane could be changed to a different porosity in order to increase the capillary flow of the sample.

Alternate 6

The goat anti-CRP antibody could be produced in other animal species, not specifically goat.

Alternate 7

The dilution of the sample could be variable to accommodate different sensitivities or detection limits.

Alternate 8

The dilution buffer could consist of any buffer that resulted in an accurate test. Shelf life could be extended by the addition of preservatives but not limited to sodium azide.

Alternate 9

The dilution buffer could be eliminated and sample could be applied neat. This would require a change in all of the concentrations of the reagents.

Alternate 10

The anti-CRP antibody could be coupled to latex beads rather than the colloidal gold. Since the purpose of the colloidal gold is to provide a visualization of the reaction, any latex particle that would allow visualization could be used.

Alternate 11

The test could easily be adapted to a dipstick format rather than a lateral flow assay requiring a housing. Rather than applying the sample into the sample well, the dipstick would be briefly immersed in the sample. The dipstick consists of the lateral flow strip without the plastic housing. It is assembled with adhesive cover tape to maintain flow characteristics.

Alternate 12

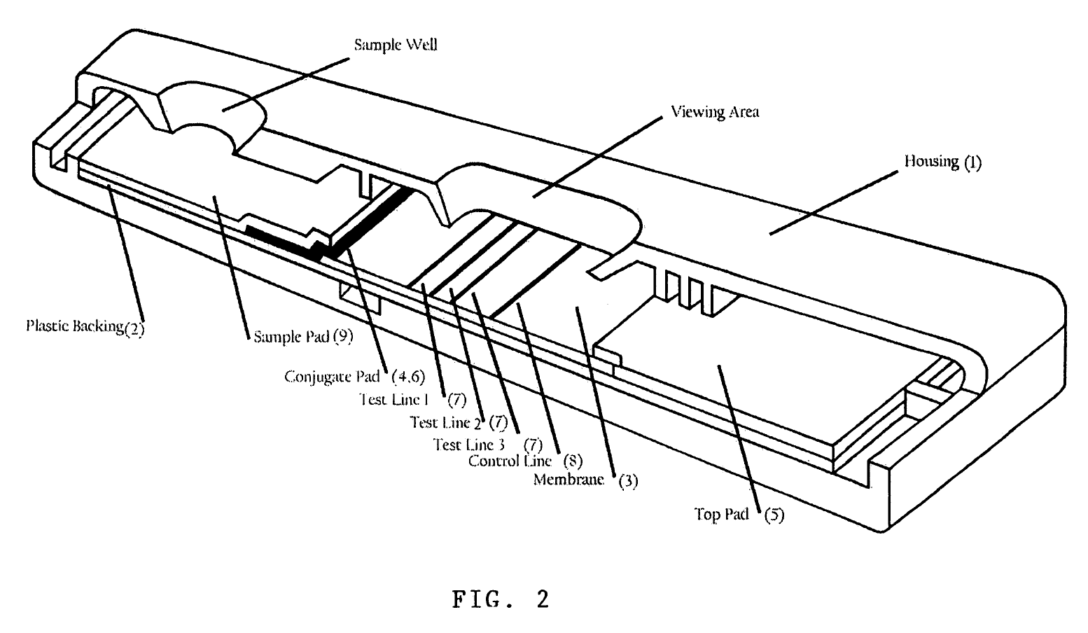

The test could also include multiple lines aimed at detecting several concentrations of CRP in the sample as illustrated in FIG. 2. This could potentially be used to make a semi-quantitative device.

### Appendix

1. Ridker, P. 2001. High-sensitivity C-Reactive Protein; Potential adjunct for global risk assessment in the primary prevention of cardiovascular disease. Circulation. 103:1813-1818.

2. Ridker, P. 1999. Evaluation novel cardiovascular risk factors:can we better predict heart attacks? Ann. Intern. Med. 130:933-937.

3. Lagrand, W., et al. 1999. C-reactive protein as a cardiovascular risk factor: more than an epiphenomenon? Circulation. 100:96-102.

4. Ridker, P., et al. 2000. C-reactive protein and other markers of inflammation in the prediction of cardiovascular disease in women. N. Engl. J Med. 342:836-843.

5. Kuller, L., et al. 1996. Relationship of C-reactive protein and coronary heart disease in the MRFIT nested case-control study. Am. J Epidemiol. 144:537-547.

6. Ridker, P., et al. 1997. Inflammation, aspirin, and the risk of cardiovascular disease in apparently healthy men. N. Engl. J Med. 336:973-979.

7. Tracy, R., et al. 1997. Relationship of C-reactive protein to risk of cardiovascular disease in the elderly: results from the Cardiovascular Health Study and the Rural Health Promotion Project. Arterioscler. Thromb. Vasc. Biol. 17:1121-1127.

8. Ridker, P., et al. 1998. Prospective study of C-reactive protein and the risk of future cardiovascular events among apparently healthy women. Circulation. 98:731-733.

9. Ridker, P., et al. 1998. Plasma concentration of C-reactive protein and risk of developing peripheral vascular disease. Circulation. 97:425-428.

10. Koenig, W., et al. 1999. C-reactive protein, a sensitive marker of inflammation, predicts future risk of coronary heart disease in initially healthy middle-aged men: results from the MONICA (MONItoring trends and determinants in CArdiovascular disease) Augsberg Cohort Study, 1984 to 1992). Circulation. 99:237-242.

11. Roivainen, M. et al. 2000. Infections, inflammation, and the risk of coronary heart disease. Circulation. 101:252-257.

12. Harris, T., et al. 1999. Associations of elevated interleukin-6 and C-reactive protein levels with mortality in the elderly. Am. J Med. 106:506-512.

13. Danesh, J., et al. 2000. Low grade inflammation and coronary heart disease: prospective study and updated meta-analyses. BMJ. 321:199-204.

14. Mendall, M., et al. 2000. C-reactive protein: relation to total mortality, cardiovascular mortality and cardiovascular risk factors in men. Eur. Heart J 21:1584-1590.

15. Ledue, T., Rifai, N. 2003. Preanalytic and analytic sources of variations in C-reactive protein measurement: implications for cardiovascular disease risk assessment. Clin. Chem. 49:1258-1271.

16. Pearson, T., et al. 2003. Markers of Inflammation and Cardiovascular Disease: Application to Clinical and Public Health Practice: A Statement for Healthcare Professionals From the Centers for Disease Control and Prevention and the American Heart Association. Circulation. 107:499-511.

1. A method of determining if the concentration of CRP in biological fluids of a mammal is above a predetermined relative risk level, the method comprising the steps of:
taking a sample of a biological fluid from the mammal;
exposing a quantity of the sample to a quantity of a dehydrated anti-CRP complexing agent coupled with a labeling agent that is immobilized at a sample position on a protein binding membrane to yield a conjugate;
exposing the conjugate to a quantity of a dehydrated standard mammalian anti-CRP antibody immobilized at a test position on the membrane, the test position being spaced from the sample position on the membrane; and
observing binding of the anti-CRP complexing agent with the standard mammalian anti-CRP antibody evidenced by a color change from the labeling agent at a test position which color change indicates that the concentration of CRP is above the predetermined threshold level.

2. The method of claim 1, further including the steps of:
exposing the conjugate to a quantity of dehydrated protein immobilized at a control position on the membrane, the control position being spaced from the test position; and
observing the binding of the anti-CRP complexing agent and the protein evidenced by a color change from the labeling agent at a control position.

3. The method of claim 1, further including the step of: diluting the sample before exposing it to the anti-CRP complexing agent.

4. The method of claim 2, further including the step of: diluting the sample before exposing it to the anti-CRP complexing agent.

5. The method of claim 1, wherein the anti-CRP complexing agent and standard mammalian anti-CRP antibody are applied and dried in spaced relationship on a protein binding membrane.

6. The method of claim 2, wherein the protein is immobilized on a protein binding membrane.

7. The method of claim 5, wherein the membrane is formed of a material selected form the group consisting of polyvinylidene difluoride, mixed cellulose ester, and nitrocellulose.

8. The method of claim 6, wherein the membrane is formed of a material selected from the group consisting of polyvinylidene difluoride, mixed cellulose ester, and nitrocellulose.

9. The method of claim 1, wherein the biological fluid is selected from a group consisting of whole blood, plasma, serum, colostrum, and urine.

10. The method of claim 1, wherein the mammal is selected from a group consisting of cats, cattle, dogs, horses, humans and swine.

11. The method of claim 1, wherein the predetermined threshold level ranges from about 1 mg/L to about 4 mg/L.

12. The method of claim 11, wherein the predetermined threshold level is about 1.5 mg/L.

13. The method of claim 2, wherein the protein is selected from a group consisting of native Protein A, native Protein G, recombinant Protein A, recombinant Protein G, and mixtures thereof.

14. The method of claim 1, wherein the anti-CRP complexing agent is coupled with a labeling agent selected from a group consisting of colloidal gold and latex particles.

15. The method of claim 5, wherein the membrane is supported in a lateral flow cassette, and wherein the sample is deposited on the membrane through an opening in the cassette.

16. The method of claim 5, wherein the membrane is supported on a dipstick structure, and wherein the sample is deposited on the membrane by placing the dipstick structure into the sample.

17. The method of claim 5, wherein a first quantity of standard mammalian anti-CRP antibody is immobilized at a first test position on the membrane, and a second quantity of standard mammalian anti-CRP antibody is immobilized at a second test position spaced from the first test position, wherein binding of the anti-CRP complexing agent with the first and second quantities of standard mammalian anti-CRP antibody is evidenced by color changes at the first and second test positions.

18. The method of claim 17, wherein a third quantity of standard mammalian anti-CRP antibody is immobilized at a third test position on the membrane, and wherein binding of the anti-CRP complexing agent with the third quantity of standard mammalian anti-CRP antibody is evidenced by a color change at the third test position.
